SEQUENCE LISTING (17 sequences)

SEQ ID NO: 1 - DNA, 20 bp, Sus scrofa
ggtagtactcacgaacactc

SEQ ID NO: 2 - DNA, 20 bp, Sus scrofa
ctaccctttcttgcccagag

SEQ ID NO: 3 - DNA, 8868 bp, Artificial Sequence
  The targeting vector of  beta4GalNT2-CRISPR
atgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtagtactcacgaacactcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctaccctttcttgcccagaggttttagagctagaaatagcaagttaaaataaggctagtccgttatcaacttgaaaaagtggcaccgagtcggtgcttttttttactagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctc

SEQ ID NO: 4 - DNA, 8505 bp, Artificial Sequence
  The targeting vector of  GGTA1-CRISPR/Cas9
tgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaaaataatgaatgtcaagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctc

SEQ ID NO: 5 - DNA, 8508 bp, Artificial Sequence
  The targeting vector of  CMAH-CRISPR/Cas9
atgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtaaggtacgtgatctgtgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctc

SEQ ID NO: 6 - RNA, 20 nt, Artificial Sequence
  beta4GalNT2/sgRNA1
gguaguacucacgaacacuc

SEQ ID NO: 7 - RNA, 20 nt, Artificial Sequence
  beta4GalNT2/sgRNA2
cuacccuuucuugcccagag

SEQ ID NO: 8 - DNA, 20 bp, Artificial Sequence
  beta4GalNT2 reverse primer
cgccgtgtaaagaaacctcc

SEQ ID NO: 9 - RNA, 18 nt, Artificial Sequence
  GGTA1/sgRNA
gaaaauaaugaaugucaa

SEQ ID NO: 10 - RNA, 20 nt, Artificial Sequence
  CMAH/sgRNA
gaguaagguacgugaucugu

SEQ ID NO: 11 - DNA, 25 bp, Artificial Sequence
  GGTA1 forward primer
ccttagtatccttcccaacccagac

SEQ ID NO: 12 - DNA, 26 bp, Artificial Sequence
  GGTA1 reverse primer
gctttctttacggtgtcagtgaatcc

SEQ ID NO: 13 - DNA, 22 bp, Artificial Sequence
  CMAH forward primer
cttggaggtgatttgagttggg

SEQ ID NO: 14 - DNA, 22 bp, Artificial Sequence
  CMAH reverse primer
cattttcttcggagttgagggc

SEQ ID NO: 15 - DNA, 18 bp, Artificial Sequence
  beta4GalNT2 forward primer
cccaaggatcctgctgcc

SEQ ID NO: 16 - RNA, 20 nt, Artificial Sequence
  beta4GalNT2/sgRNA1 or beta4GalNT2/sgRNA2
  misc_feature (1)..(1): n is c or g
  misc_feature (2)..(2): n is g or u
  misc_feature (3)..(3): n is a or u
  misc_feature (4)..(4): n is a or c
  misc_feature (5)..(5): n is c or g
  misc_feature (6)..(6): n is c or u
  misc_feature (7)..(7): n is a or u
  misc_feature (8)..(8): n is c or u
  misc_feature (11)..(11): n is a or u
  misc_feature (12)..(12): n is c or u
  misc_feature (14)..(14): n is a or c
  misc_feature (15)..(15): n is a or c
  misc_feature (18)..(18): n is c or g
  misc_feature (19)..(19): n is a or u
  misc_feature (20)..(20): n is c or g
nnnnnnnnucnngnncannn

SEQ ID NO: 17 - RNA, 80 nt, Artificial Sequence
  Skeletal sequence
guuuuagagcuagaaauagcaaguuaaaauaaggcuaguccguuaucaacuugaaaaaguggcaccgagucggugcuuuu

(19) United States
(12) Patent Application Publication
(10) Pub. No.: US 2023/0174983 A1
(43) Pub. Date: Jun. 8, 2023

(54) GRNA FOR KNOCKING OUT PIG XENOANTIGEN GENE, AND APPLICATION THEREOF

(71) Applicant: NANJING GENEFRIEND-BIOTECH INC., Nanjing, Jiangsu (CN)

(72) Inventors: Yifan DAI, Nanjing, Jiangsu (CN); Haiyuan YANG, Nanjing, Jiangsu (CN)

(21) Appl. No.: 17/921,809

(22) PCT Filed: Jul. 13, 2021
(86) PCT No.: PCT/CN2021/105973

§ 371 Date: Oct. 27, 2022

(30) Foreign Application Priority Data
Jul. 14, 2020 (CN) 202010676389.7

(51) Int. Cl. C12N 15/113; C12N 15/85; A01K 67/027; C12N 9/10; C12N 9/22

(52) U.S. Cl. CPC C12N 15/113; C12N 15/85; A01K 67/0276; C12N 9/1051; C12N 9/22; A01K 2217/075; A01K 2227/108; A01K 2267/03; A01K 2267/0387; C12N 2310/10

(57) ABSTRACT

Provided is gRNA specifically targeting β4GalNT2 gene. The gRNA specifically binds to the nucleotide sequence shown in any one of SEQ ID NOs. 1 and 2. Also provided are an animal model constructed using the gRNA, and an application thereof in the field of biomedicine.

### FIELD OF THE INVENTION

The present application relates to the field of biomedicine, and in particular, relates to a gRNA specifically targeting a β4GalNT2 gene, and an application thereof.

### BACKGROUND OF THE INVENTION

At present, cell, tissue and/or organ transplantation is a conventional option for the treatment of various diseases, including kidney, heart, lung, liver and other organ diseases, or skin damage. To overcome the challenges arising from insufficient grafts and unmet clinical need, xenotransplantation has emerged for the transplantation of a graft derived from one species (for example, pig) into another species (for example, human). However, the use of a standard unmodified heterologous graft may be associated with severe immune rejection.

To address immune rejection associated with existing xenotransplantation therapies, genes encoding some proteins that induce immune system responses may be knocked out by gene editing.

### SUMMARY OF THE INVENTION

The present application provides a gRNA specifically targeting a β4GalNT2 gene, wherein the said gRNA comprises a nucleotide sequence as set forth in any one of SEQ ID NOs. 6-7. The gRNA defined in the present application significantly improves a gene knockout efficiency. With said gRNA specifically targeting the β4GalNT2 gene in the present application, the knockout efficiency for said β4GalNT2 gene can reach at least 20% or more (for example, at least 21% or more, 22% or more, 23% or more, 24% or more, 25% or more, 26% or more, 27% or more, 28% or more, 29% or more or higher). The knockout efficiency can be improved by, for example, at least 5% or more (for example, at least 10% or more, 15% or more, 20% or more, 25% or more, 30% or more, 35% or more, 40% or more, 45% or more, 50% or more or higher) by using two sgRNAs as compared to by using one gRNA. Said gRNA specifically targeting the β4GalNT2 gene in the present application may also be used for gene knockout together with a gRNA specifically targeting a GGTA1 gene and/or a CMAH gene, such that the efficiency of simultaneous knockout for the three genes can reach at least 10% or more (for example, at least 11% or more, 12% or more, 13% or more, 14% or more, 15% or more, 16% or more, 17% or more, 18% or more, 19% or more, 20% or more, 25% or more or higher). The sgRNA defined in the present application may achieve a high knockout efficiency and/or a stable knockout efficiency.

The present application further provides a nucleic acid molecule encoding said gRNA, a cell, vector, vector transcript, kit and/or system comprising said nucleic acid molecule or sgRNA, and/or an animal model, cell, tissue and/or organ prepared according to said gRNA, and applications thereof.

In one aspect, the present application provides a gRNA specifically targeting a β4GalNT2 gene, wherein said gRNA specifically binds to a nucleotide sequence as set forth in any one of SEQ ID NOs. 1-2.

In some embodiments, said gRNA comprises a nucleotide sequence as set forth in SEQ ID NO. 16.

In some embodiments, said gRNA comprises a nucleotide sequence as set forth in any one of SEQ ID NOs. 6-7.

In some embodiments, said sgRNA comprises 5′-(X)n-said nucleotide sequence as set forth in SEQ ID NO.16-a backbone sequence-3′, with X being a base selected from any one of A, U, C, and G, and n being any integer from 0 to 15.

In some embodiments, said gRNA is a single-strand guide RNA (sgRNA).

In another aspect, the present application provides a gRNA combination, comprising said gRNA specifically targeting the β4GalNT2 gene in the present application, a gRNA specifically targeting a GGTA1 gene, and a gRNA specifically targeting a CMAH gene.

In some embodiments, said gRNA specifically targeting the GGTA1 gene comprises a nucleotide sequence as set forth in SEQ ID NO. 9.

In some embodiments, said gRNA specifically targeting the CMAH gene comprises a nucleotide sequence as set forth in SEQ ID NO. 10.

In another aspect, the present application provides an isolated nucleic acid molecule or isolated nucleic acid molecules, encoding said gRNA specifically targeting the β4GalNT2 gene in the present application.

In another aspect, the present application provides a vector comprising said nucleic acid molecule of the present application.

In some embodiments, according to the present application, one and/or two nucleic acid molecules encoding said gRNA specifically targeting the β4GalNT2 gene, a nucleic acid molecule encoding said gRNA specifically targeting the GGTA1 gene, and a nucleic acid molecule encoding said gRNA specifically targeting the CMAH gene are located in the same vector.

In another aspect, the present application provides a cell comprising said gRNA specifically targeting the β4GalNT2 gene, said nucleic acid molecule, said vector, and/or an in vitro transcription product of the vector.

In another aspect, the present application provides use of said gRNA specifically targeting the β4GalNT2 gene, said nucleic acid molecule, said vector, an in vitro transcription product of said vector and/or said cell in knocking out said β4GalNT2 gene, or in constructing an animal model.

In another aspect, the present application provides a β4GalNT2 gene-deleted cell line prepared by using said sgRNA specifically targeting the β4GalNT2 gene, said nucleic acid molecule, said vector, an in vitro transcription product of said vector and/or said cell.

In another aspect, the present application provides a method for constructing an animal model, comprising administering at least two gRNAs specifically targeting a β4GalNT2 gene to an animal cell to knock out all or a portion of said β4GalNT2 gene, wherein said gRNA specifically binds to a nucleotide sequence as set forth in any one of SEQ ID NOs: 1-2.

In some embodiments, said gRNA specifically targeting the β4GalNT2 gene comprises a nucleotide sequence as set forth in SEQ ID NO. 16.

In some embodiments, said gRNA specifically targeting the β4GalNT2 gene comprises a nucleotide sequence as set forth in any one of SEQ ID NOs. 6-7.

In some embodiments, said gRNA specifically targeting the β4GalNT2 gene comprises 5′-(X)n-said nucleotide sequence as set forth in SEQ ID NO.16-a backbone sequence-3′, with X being a base selected from any one of A, U, C, and G, and n being any integer from 0 to 15.

In some embodiments, said gRNA is a single-strand guide RNA (sgRNA).

In some embodiments, said method comprises the step of producing one or more single-strand breaks (SSBs) or double-strand breaks (DSBs) within or near said β4GalNT2 gene by using one or more deoxyribonucleic acid (DNA) endonucleases, such that all or a portion of one or more exons of said β4GalNT2 gene are/is deleted.

In some embodiments, said method further comprises administering a gRNA specifically targeting a GGTA1 gene to an animal cell to knock out all or a portion of said GGTA1 gene.

In some embodiments, said gRNA specifically targeting the GGTA1 gene comprises a nucleotide sequence as set forth in SEQ ID NO. 9.

In some embodiments, said method further comprises administering a gRNA specifically targeting a CMAH gene to an animal cell to knock out all or a portion of said CMAH gene.

In some embodiments, said gRNA specifically targeting the CMAH gene comprises a nucleotide sequence as set forth in SEQ ID NO. 10.

In some embodiments, said method comprises the step of producing one or more single-strand breaks (SSBs) or double-strand breaks (DSBs) within or near said GGTA1 gene and/or said CMAH gene by using one or more deoxyribonucleic acid (DNA) endonucleases, such that all or a portion of one or more exons of said GGTA1 gene and/or said CMAH gene is/are deleted.

In some embodiments, said DNA endonuclease comprises a Cas nuclease.

In some embodiments, said Cas nuclease comprises a Cas9 nuclease, a homologue thereof, a recombinant of a naturally occurring molecule thereof, a codon-optimized version thereof, and/or a modified version thereof.

In some embodiments, said method comprises: a) providing a cell comprising one or more in vitro transcription products comprising said vector of the present application or a vector of said gRNA; b) culturing said cell in a culture solution; c) transplanting said cultured cell into a fallopian tube of a receptor female non-human mammal, to allow said cell to develop in a uterus of said female non-human mammal; and d) identifying germline transmission in a genetically modified non-human mammal of a progeny of said pregnant female of step c).

In some embodiments, said animal comprises a pig.

In another aspect, the present application provides an animal model prepared according to said method for constructing an animal model in the present application, wherein said animal does not express said β4GalNT2 gene and/or β-1,4-N-acetylgalactosaminyl transferase 2.

In some embodiments, said animal does not express said GGTA1 gene and/or αGal.

In some embodiments, said animal does not express said CMAH gene and/or Neu5Gc.

In another aspect, the present application provides a method for preparing an animal model, comprising: a) providing said animal model of the present application; and b) mating said animal model obtained in step a) with another animal, or performing in vitro fertilization on said animal model obtained in step a), or further performing gene editing on said animal model obtained based on step a), or transplanting a human tissue and cell into said animal model obtained in step a), and performing screening to obtain an animal model.

In another aspect, the present application provides an animal model prepared according to said method for preparing an animal model.

In some embodiments, said animal comprises a pig.

In another aspect, the present application provides a cell or cell line or primary cell culture, wherein said cell or cell line or primary cell culture is derived from said animal model of the present application or a progeny thereof.

In another aspect, the present application provides a tissue or organ or a culture thereof, wherein said tissue or organ or the culture thereof is derived from said animal model or a progeny thereof.

In another aspect, the present application provides a CRISPR/Cas9 system for specifically targeting and knocking out a β4GalNT2 gene. The CRISPR/Cas9 system comprises the use of a DNA sequence containing said gRNA specifically targeting the β4GalNT2 gene in the present application.

In another aspect, the present application provides a nucleic acid molecule kit capable of specifically targeting a β4GalNT2 gene, wherein said kit comprises said gRNA specifically targeting the β4GalNT2 gene.

In another aspect, the present application provides a complete set of nucleic acid molecules capable of specifically targeting a β4GalNT2 gene, wherein said complete set of nucleic acid molecules comprises said sgRNA specifically targeting the β4GalNT2 gene and a Cas9 mRNA.

In another aspect, the present application provides an application of said cell or cell line or primary cell culture or said tissue or organ or the culture thereof in development of an organ and/or tissue transplantation product, or as a model system for pharmacological, immunological and medical researches, or in validation, evaluation or study of immune rejection.

In another aspect, the present application provides an application of said animal model in development of an organ and/or tissue transplantation product, or as a model system for pharmacological, immunological and medical researches.

In another aspect, the present application provides an application of said animal model in the aspect of validating, evaluating or studying immune rejection.

Other aspects and advantages of the present application can be readily perceived by those skilled in the art from the detailed description below. The detailed description below only shows and describes the exemplary embodiments of the present application. As would be appreciated by those skilled in the art, the content of the present application allows those killed in the art to change the specific embodiments disclosed without departing from the spirit and scope involved in the present application. Accordingly, the accompanying drawings and the description in the specification of the present application are merely for an exemplary but not restrictive purpose.

### BRIEF DESCRIPTION OF THE DRAWINGS

The specific features of the present invention involved in the present application are listed in the appended claims. The characteristics and advantages of the present invention involved in the present application can be better understood by referring to the exemplary embodiments and the accompanying drawings described in detail below. A brief description of the drawings is as follows:

FIG. 1 shows a schematic diagram of a GGTA1-CRISPR/Cas9 targeting vector;

FIG. 2 shows a schematic diagram of a CMAH-CRISPR/Cas9 targeting vector; and

FIG. 3 shows a schematic diagram of a double-sgRNA β4GalNT2-CRISPR/Cas targeting vector.

### DETAILED DESCRIPTION OF THE PREFERRED EMBODIMENTS

The embodiments of the invention of the present application will be illustrated by specific examples below. Those familiar with this technology can easily understand other advantages and effects of the invention of the present application from the content disclosed in the specification.

### Terms & Definitions

In the present application, the term “single-strand break (SSB)” generally refers to a break caused by cleavage in one strand of a DNA molecule. When only one of the two strands of the DNA double helix is defective, the other strand can be used as a template to guide the correction of the damaged strand. A DNA endonuclease may cause the single-strand break.

In the present application, the term “double-strand break (DSB)” generally refers to a phenomenon that occurs when two single strands of a double-strand DNA molecule are cleaved at the same location. The double-strand break can induce DNA repair, possibly leading to genetic recombination, and cells also have some systems that act on a double-strand break that occurs at other times. The double-strand break may occur periodically during the normal cell replication cycle, or may be enhanced in some cases with, for example, ultraviolet light, and DNA break inducers (for example, various chemical inducers). Many inducers can induce DSBs to occur indiscriminately across the genome, and the DSBs can be regularly induced and repaired in normal cells. During repair, an original sequence can be reconstructed with full fidelity. However, in some cases, small insertions or deletions (called “indels”) are introduced at DSB sites. In some cases, the double-strand break can also be specifically induced at a specific location, which can contribute to targeted or preferential genetic modification at a selected chromosomal location. In many cases, the trend of recombination of homologous sequences during DNA repair (and replication) can also be exploited, which underlies the application of a gene editing systems (such as CRISPR). This homology-guided repair is used for inserting a sequence of interest provided by using a “donor” polynucleotide into a desired chromosomal location.

In the present application, the term “in vitro transcription product” generally refers to a product that is synthesized by in vitro transcription of DNA, or that is processed and then becomes a messenger RNA (mRNA). The in vitro transcription product may comprise a precursor messenger RNA (pre-mRNA) and the processed mRNA itself. After DNA strands are transcribed into transcripts, newly synthesized primary transcripts can be modified in several ways and converted into to their mature functional forms, so as to produce different proteins and RNAs (such as mRNA, tRNA, rRNA, lncRNA, miRNA, etc.). The term “in vitro transcript” may comprise an exon, an intron, 5′UTR and 3′UTR.

In the present application, the “vector” generally refers to a nucleic acid molecule capable of self-replication in a suitable host, and it is used to transfer an inserted nucleic acid molecule into and/or between host cells. The vector may include a vector mainly for inserting DNA or RNA into cells, a vector mainly for replicating DNA or RNA, and a vector mainly for expressing DNA or RNA transcription and/or translation. The vector also includes a carrier having a variety of the functions defined above. The vector may be a polynucleotide that may be transcribed and translated into a polypeptide when introduced into a suitable host cell. Generally, the vector may produce a desired expression product by culturing a suitable host cell containing the vector. The vector may encompass the following additional features in addition to a transgene insertion sequence and a backbone: a promoter, a genetic marker, antibiotic resistance, a reporter gene, a targeting sequence, and a protein purification tag. A vector called an expression vector (expression construct) is used specifically to express a transgene in a target cell, and generally has a control sequence. The vector described in the present application may be an expression vector, which may comprise a viral vector (a lentiviral vector and/or a retroviral vector), a phage vector, a phagemid, a cosmid, an artificial chromosome such as a yeast artificial chromosome (YAC), a bacterial artificial chromosome (BAC) or a P1-derived artificial chromosome (PAC), and/or a plasmid.

In the present application, the term “pig” generally refers to any pig known in the art, including but not limited to: a wild pig, a domestic pig, a mini pig, a Sus scrofa pig, a Sus scrofa domesticus pig, and an inbred pig. Without limitation, the pig may be selected from a group consisting of: a Landrace pig, a Yorkshire pig, a Hampshire pig, a Duroc pig, a Chinese Meishan pig, a Chester white pig, a Berkshire Goettingen pig, a Landrace/Yorkshire/Chester pig, a Yueatan pig, a Barna pig, a Wuzhishan pig, a Xi Shuang Banna pig, and a Pietrain pig.

In the present application, the term “deoxyribonucleic acid (DNA) endonuclease” generally refers to an enzyme capable of hydrolyzing a phosphodiester bond inside a DNA molecular chain to produce an oligonucleotide. The DNA endonucleases may comprise enzymes having no base specificity and enzymes capable of recognizing and cleaving specific bases or base sequences.

In the present application, the term “Cas nuclease” generally refers to a CRISPR-associated nuclease, which is a type of DNA endonuclease capable of causing double-strand breaks at a specific DNA sequence.

The Cas nuclease can be generally complementary to a CRISPR sequence and can take the CRISPR sequence as a guide to recognize and cleave a specific DNA strand. Examples of the Cas nucleases may comprise, but are not limited to, a group consisting of: Casl, CaslB, Cas2, Cas3, Cas4, Cas5, Cas6, Cas7, Cas8, Cas9 (also known as Csnl and Csxl2), CaslO, Csyl, Csy2, Csy3, Csel, Cse2, Csc1, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csx14, CsxlO, Csxl6, CsaX, Csx3, Csxl, Csxl5, Csfl, Csf2, Csfl, Csf4, and/or their homologues, or modified forms thereof.

In the present application, the term “Cas9 nuclease” may also be called Csn1 or Csxl2, and generally refers to an RNA-guided DNA endonuclease associated with a type II clustered regularly interspaced short palindromic repeat (CRISPR) adaptive immune system. The Cas9 nuclease can also comprise a wild-type protein, an ortholog, and functional and non-functional mutants thereof. The Cas9 nuclease can be derived from any suitable bacteria. The Cas9 nuclease generally comprises a RuvC nuclease domain and an HNH nuclease domain, which cleave two different strands of a double-stranded DNA molecule, respectively. The Cas9 nuclease has been described in connection to different bacterial species such as S. thermophiles, Listeria innocua (Gasiunas, Barrangou et al., 2012; Jinek, Chylinski et al., 2012) and S. pyogenes (Deltcheva, Chylinski et al. 2011), for example, a Streptococcus pyogenes Cas9 protein, whose amino acid sequence can be found in SwissProt database under Accession Number Q99ZW2; a Neisseria meningitides Cas9 protein, whose amino acid sequence can be found in UniProt database under Number A1IQ68; a Streptococcus thermophiles Cas9 protein, whose amino acid sequence can be found in UniProt database under Number Q03LF7; and a Staphylococcus aureus Cas9 protein, whose amino acid sequence can be found in UniProt database under Number J7RUA5.

In the present application, the term “gRNA” generally refers to a guide RNA, which is a type of RNA molecule. In the nature, crRNA and tracrRNA are usually present as two separate RNA molecules to constitute a gRNA. The term “crRNA”, also known as CRISPRRNA, generally refers to a nucleotide sequence complementary to a targeted DNA of interest, and the term “tracrRNA” generally refers to a scaffold-type RNA that is bindable to a Cas nuclease. The crRNA and the tracRNA can also be fused into a single strand, and in this case, the gRNA may also be called a single-stranded guide RNA (sgRNA), which has become the most common form of gRNA used in CRISPR technologies by those skilled in the art. Therefore, the terms “sgRNA” and “gRNA” may have the same meaning herein. The sgRNA may be artificially synthesized, or prepared from a DNA template in vitro or in vivo. The sgRNA can bind to the Cas nuclease or target a DNA of interest. It can guide the Cas nuclease to cleave a DNA site complementary to the gRNA. The degree of complementation between the gRNA and its corresponding target sequence is at least about 50%.

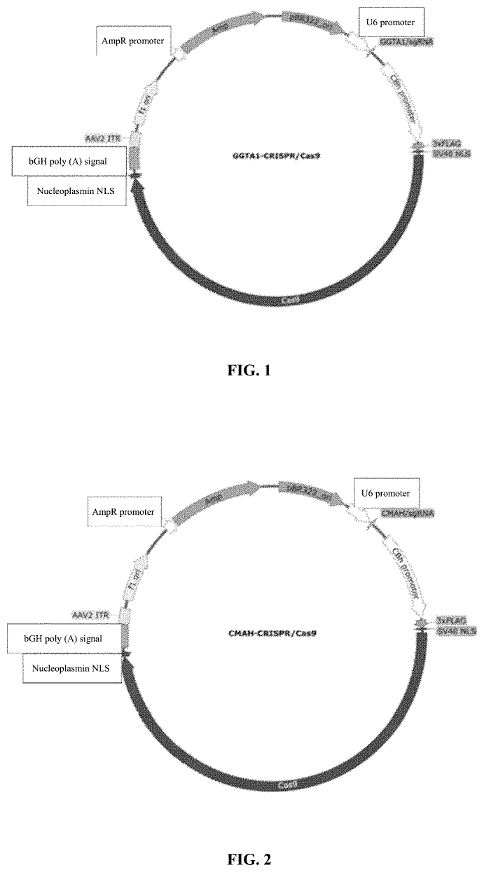

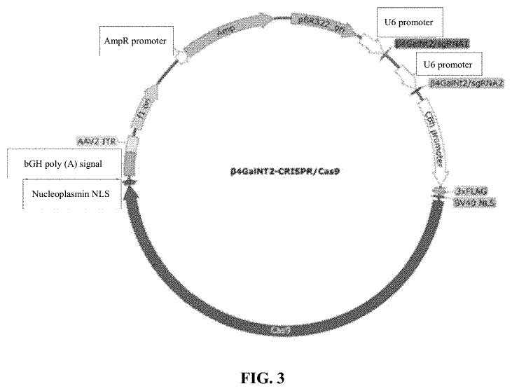

In the present application, the term “backbone sequence” generally refers to a portion of the gRNA, other than the portion that recognizes or hybridizes with a target sequence, and it may comprise a sequence, in the sgRNA, between a gRNA pairing sequence and a transcription terminator. The backbone sequence generally does not change due to a change in the target sequence, nor does it affect the recognition of the target sequence by the gRNA. Thus, the backbone sequence may be any sequence available in the prior art. The structure of the backbone sequence can be found in, for example, A and B in FIG. 1, A, B and C in FIG. 3, and A, B, C, D and E in FIG. 4, except a portion described a spacer sequence, in Nowak et al. Nucleic Acids Research 2016.44:9555-9564.

In the present application, the terms “target nucleic acid”, “target nucleic acid” and “target region” are used interchangeably, and generally refer to a nucleic acid sequence recognizable by a gRNA. The target nucleic acid can refer to a double-stranded nucleic acid or a single-stranded nucleic acid.

In the present application, the term “isolated nucleic acid molecule” generally refers to a single-stranded or double-stranded polymer of a deoxyribonucleotide or ribonucleotide base, or an analog thereof, read from a 5′-terminal to a 3′-terminal. The isolated nucleic acid molecule can be isolated from a normal or natural environment, or can be produced by artificial synthesis. Such an isolated nucleic acid molecule is removed or isolated from its normal or natural environment, or is produced in such a way that it is not present in its normal or natural environment, thereby isolating it from polypeptides, peptides, lipids, carbohydrates, other polynucleotides or other materials in its normal or natural environment. The isolated nucleic acid molecule in the present application may encode an RNA, for example, a gRNA specifically targeting a RPGR gene.

In the present application, the term “CMAH” generally refers to a gene encoding cytidine monophosphate-N-acetylneuraminic acid hydroxylase (CMP-Neu5Ac hydroxylase). The functional cytidine monophosphate-N-acetylneuraminic acid hydroxylase can catalyze the conversion of sialic acid N-acetylneuraminic acid (Neu5Ac) to N-hydroxyacetylneuraminic acid (Neu5Gc). A Neu5Gc residue is an epitope or antigen that is recognized by a human immune system. The cytidine monophosphate-N-acetylneuraminic acid hydroxylases described in the present application may comprise a full-length protein, a functional fragment, a homologue, and/or a functional variant (such as a splice variant). A nucleotide sequence of the CMAH gene described in the present application comprises its functional variant, derivative, analog, homologue and a fragment thereof. The amino acid sequence of a pig CMAH protein can be found in the NCBI database under Accession Number NP_001106486.1, and a pig CMAH nucleotide sequence can be found in the NCBI database under Accession Number NM_001113015.1.

In the present application, the term “GGTA1” may also be called αGal, GGTA, GGT1, GT, aGT or GGTA1, and generally refers to a gene encoding α1,3 galactosyltransferase (αGal, GT). A functional α1,3 galactosyltransferase can catalyze the formation of a galactose α1,3-galactose (αGal, Gal, Gal, gall, 3gal, or gal1-3gal) residue on a glycoprotein. The galactose α1,3-galactose (αGal) residue is an epitope or antigen that is recognized by the human immune system. The α1,3 galactosyltransferase described in the present application may comprise a full-length protein, a functional fragment, a homologue, and/or a functional variant (such as a splice variant). The nucleotide sequence of the GGTA1 gene described in the present application comprises its functional variant, derivative, analog, homologue and a fragment thereof. The amino acid sequence of a pig GGTA1 protein can be found in the NCBI database under Accession Number NP_001309984.1, and a pig GGTA1 nucleotide sequence can be found in the NCBI database under Accession Number NM_001323055.1.

In the present application, the term “β4GalNT2” generally refers to a gene encoding β-1,4N-acetylgalactosaminyl transferase 2 (β4GalNT2, β4GalNT2, β1,4GalNT2, β1,4GalNT2), and a functional β4GalNT2 can produce a Sda-like glycan. The β-1,4N-acetylgalactosaminyl transferase 2 described in the present application may comprise a full-length protein, a functional fragment, a homologue, and/or a functional variant (such as a splice variant). The nucleotide sequence of the β4GalNT2 gene described in the present application comprises its functional variant, derivative, analog, homologue and a fragment thereof. The amino acid sequence of the pig β4GalNT2 protein can be found in the NCBI database under Accession Number NP_001231259.1, and the pig β4GalNT2 nucleotide sequence can be found in the NCBI database under Accession Number NM_001244330.1.

### DETAILED DESCRIPTION OF THE INVENTION

In one aspect, the present application provides a gRNA specifically targeting a β4GalNT2 gene, which may specifically bind to a nucleotide sequence as set forth in any one of SEQ ID NOs. 1-2. In some cases, the gRNA specifically targeting the β4GalNT2 gene may specifically bind to a nucleotide sequence having at least 70% (for example, at least 75%, at least 80%, at least 85%, at least 90%, at least 91%, at least 92%, at least 93%, at least 94%, at least 95%, at least 96%, at least 97%, at least 98%, at least 99%, or at least 100%) sequence identity to a nucleotide sequence as set forth in any one of SEQ ID NOs. 1-2.

In some cases, the gRNA specifically targeting the β4GalNT2 gene may specifically bind to a nucleotide sequence complementary to a nucleotide sequence as set forth in any one of SEQ ID NOs. 1-2. In some cases, the gRNA may specifically bind to a nucleotide sequence complementary to the nucleotide sequence having at least 70% (for example, at least 75%, at least 80%, at least 85%, at least 90%, at least 91%, at least 92%, at least 93%, at least 94%, at least 95%, at least 96%, at least 97%, at least 98%, at least 99%, or at least 100%) sequence identity to the nucleotide sequence as set forth in any one of SEQ ID NOs. 1-2.

The gRNA specifically targeting the β4GalNT2 gene described in the present application may bind to a sequence in a target nucleic acid of interest. The gRNA may interact with the target nucleic acid in a sequence-specific form by hybridization (i.e., base pairing). A nucleotide sequence of the gRNA specifically targeting the β4GalNT2 gene may vary according to the sequence of the target nucleic acid of interest.

In the present application, the gRNA specifically targeting the β4GalNT2 gene may comprise a nucleotide sequence as set forth in SEQ ID NO. 16: n1n2n3n4n5n6n7n8tcn11n12gn14n15 can18n19n20, with n1=c or g, n2=g or u, n3=a or u, n4=a or c, n5=c or g, n6=c or u, n7=a or u, n8=c or u, n11=a or u, n12=c or u, n14=a or c, n15=a or c, n18=c or g, n19=a or u, and n20=c or g. In the present application, the gRNA may comprise a nucleotide sequence having at least 70% (for example, at least 75%, at least 80%, at least 85%, at least 90%, at least 91%, at least 92%, at least 93%, at least 94%, at least 95%, at least 96%, at least 97%, at least 98%, at least 99%, or at least 100%) sequence identity to a nucleotide sequence as set forth in SEQ ID NO 16.

In the present application, the gRNA specifically targeting the β4GalNT2 gene may comprise a nucleotide sequence as set forth in any one of SEQ ID NOs. 6-7. In the present application, the gRNA may comprise a nucleotide sequence having at least 70% (for example, at least 75%, at least 80%, at least 85%, at least 90%, at least 91%, at least 92%, at least 93%, at least 94%, at least 95%, at least 96%, at least 97%, at least 98%, at least 99%, or at least 100%) sequence identity to a nucleotide sequence as set forth in any one of SEQ ID NOs. 6-7.

In the present application, the gRNA specifically targeting the β4GalNT2 gene may comprise a backbone sequence, and the backbone sequence used in the present application may be derived from any commercially available plasmid as long as it can achieve the expression of a Cas nuclease and the transcription of a gRNA. For example, the backbone sequence may be the one derived from pX330. For example, the backbone sequence may be a nucleotide sequence as set forth in SEQ ID NO. 17. In the present application, the gRNA comprises 5′-(X)n-the nucleotide sequence as set forth in SEQ ID NO.16-a backbone sequence-3′, with X being a base selected from any one of A, U, C, and G, and n being any integer from 0 to 15. For example, n is 0. For example, the gRNA described in the present application may comprise 5′-the nucleotide sequence as set forth in SEQ ID NO: 6-the nucleotide sequence as set forth in SEQ ID NO: 17-3′, or 5′-the nucleotide sequence as set forth in SEQ ID NO: 7-the nucleotide sequence as set forth in SEQ ID NO: 17-3′.

In the present application, the gRNA specifically targeting the β4GalNT2 gene may be a single-stranded guide RNA (sgRNA).

In another aspect, the present application provides a gRNA combination, which may comprise the gRNA specifically targeting the β4GalNT2 gene in the present application, a gRNA specifically targeting a GGTA1 gene, and a gRNA specifically targeting a CMAH gene.

In the present application, the gRNA specifically targeting the GGTA1 gene may comprise a nucleotide sequence as set forth in SEQ ID NO. 9. In the present application, the gRNA specifically targeting the GGTA1 gene may comprise a nucleotide sequence having at least 70% (for example, at least 75%, at least 80%, at least 85%, at least 90%, at least 91%, at least 92%, at least 93%, at least 94%, at least 95%, at least 96%, at least 97%, at least 98%, at least 99%, or at least 100%) sequence identity to a nucleotide sequence as set forth in SEQ ID NO. 9.

In the present application, the gRNA specifically targeting the CMAH gene may comprise a nucleotide sequence as set forth in SEQ ID NO. 10. In the present application, the gRNA specifically targeting the CMAH gene may comprise a nucleotide sequence having at least 70% (for example, at least 75%, at least 80%, at least 85%, at least 90%, at least 91%, at least 92%, at least 93%, at least 94%, at least 95%, at least 96%, at least 97%, at least 98%, at least 99%, or at least 100%) sequence identity to a nucleotide sequence as set forth in SEQ ID NO. 10.

In another aspect, the present application provides an isolated nucleic acid molecule or isolated nucleic acid molecules, which may encode the gRNA specifically targeting the β4GalNT2 gene in the present application.

In another aspect, the present application provides a vector, which may comprise the nucleic acid molecule encoding the gRNA specifically targeting the β4GalNT2 gene in the present application.

When two nucleic acid molecules encoding the gRNA specifically targeting the β4GalNT2 gene in the present application are used, the two nucleic acid molecules may be located in the same vector, or in different vectors. For example, the two nucleic acid molecules encoding the gRNA specifically targeting the β4GalNT2 gene may be located in the same vector.

In the present application, one and/or two nucleic acid molecules encoding the gRNA specifically targeting the β4GalNT2 gene, a nucleic acid molecule encoding the gRNA specifically targeting the GGTA1 gene, and a nucleic acid molecule encoding the gRNA specifically targeting the CMAH gene may be each located in one, two, three or four vectors.

In the present application, one and/or two nucleic acid molecules encoding the gRNA specifically targeting the β4GalNT2 gene, the nucleic acid molecule encoding the gRNA specifically targeting the GGTA1 gene, and the nucleic acid molecule encoding the gRNA specifically targeting the CMAH gene may be each located in a different vector.

In the present application, two of one and/or two nucleic acid molecules encoding the gRNA specifically targeting the β4GalNT2 gene, a nucleic acid molecule encoding the gRNA specifically targeting the GGTA1 gene, and a nucleic acid molecule encoding the gRNA specifically targeting the CMAH gene may be located in the same vector.

In the present application, three of one and/or two nucleic acid molecules encoding the gRNA specifically targeting the β4GalNT2 gene, a nucleic acid molecule encoding the gRNA specifically targeting the GGTA1 gene, and a nucleic acid molecule encoding the gRNA specifically targeting the CMAH gene may be located in the same vector.

In the present application, one and/or two nucleic acid molecules encoding the gRNA specifically targeting the β4GalNT2 gene, a nucleic acid molecule encoding the gRNA specifically targeting the GGTA1 gene, and a nucleic acid molecule encoding the gRNA specifically targeting the CMAH gene may be located in the same vector according to the present invention.

In the present application, the vector may be any vector applicable to CRISPR/Cas. For example, the vector is a PX330 plasmid.

In another aspect, the present application provides a cell, which may comprise the gRNA specifically targeting the β4GalNT2 gene, the nucleic acid molecule, the vector, and/or an in vitro transcription product of the vector.

In another aspect, the present application provides use of the gRNA specifically targeting the β4GalNT2 gene, the nucleic acid molecule, the vector, an in vitro transcription product of the vector and/or the cell in knocking out the β4GalNT2 gene, or in constructing an animal model.

In another aspect, the present application provides a β4GalNT2 gene-deleted cell line prepared by using said sgRNA specifically targeting the β4GalNT2 gene, said nucleic acid molecule, said vector, an in vitro transcription product of said vector and/or said cell.

In another aspect, the present application provides a method for constructing an animal model. The method may comprise administering at least two gRNAs specifically targeting a β4GalNT2 gene in the present invention to an animal cell to knock out all or a portion of the β4GalNT2 gene, wherein the gRNA specifically binds to a nucleotide sequence as set forth in any one of SEQ ID NOs: 1-2.

In the present application, the method may comprise the step of producing one or more single-strand breaks (SSBs) or double-strand breaks (DSBs) within or near the β4GalNT2 gene by using one or more deoxyribonucleic acid (DNA) endonucleases, such that all or a portion of one or more exons of the β4GalNT2 gene are/is deleted.

In the present application, the method may comprise administering a gRNA specifically targeting a GGTA1 gene in the present application to an animal cell to knock out all or a portion of the GGTA1 gene.

In the present application, the method may comprise administering a gRNA specifically targeting a CMAH gene in the present application to an animal cell to knock out all or a portion of the CMAH gene.

In the present application, the method may comprise the step of producing one or more single-strand breaks (SSBs) or double-strand breaks (DSBs) within or near the GGTA1 gene and/or the CMAH gene by using one or more deoxyribonucleic acid (DNA) endonucleases, such that all or a portion of one or more exons of the GGTA1 gene and/or the CMAH gene is/are deleted.

The DNA endonuclease may comprise a deoxyribonucleic acid endonuclease I, a deoxyribonucleic acid endonuclease II, a deoxyribonucleic acid endonuclease IV, a restriction endonuclease, a UvrABC endonuclease, and/or an engineered nuclease. Examples of the engineered nuclease comprise, but is not limited to, a homing endonuclease (also known as meganuclease), a zinc finger nuclease (ZFN), a transcription activator-like effector-based nuclease (TALEN), and a clustered regularly interspaced short palindromic repeat (CRISPR).

In the present application, the DNA endonuclease may comprise a Cas nuclease. In the present application, the Cas nuclease may comprise a Cas9 nuclease, a homologue thereof, a recombinant of a naturally occurring molecule thereof, a codon-optimized version thereof, and/or a modified version thereof.

The DNA endonuclease may be modified or unmodified. Likewise, the gRNA, crRNA, tracrRNA or sgRNA may be modified or unmodified. There are many modifications that are known in the art and can be used, for example, the deletion, insertion, translocation, inactivation and/or activation of a nucleotide. These modifications may comprise introducing one or more mutations (comprising changes in single or multiple base pairs), increasing the number of hairpins, cross-linking, breaking a specific stretch of nucleotide, and other modifications. These modifications may comprise the inclusion of at least one non-naturally occurring nucleotide, or a modified nucleotide, or an analog thereof. The nucleotide may be modified at a ribose, phosphate and/or base moieties/moiety.

In the present application, the method may comprise: a) providing a cell comprising one or more in vitro transcription products comprising the vector of the present application or a vector of the gRNA; b) culturing the cell in a culture solution; c) transplanting the cultured cell into a fallopian tube of a receptor female non-human mammal, to allow the cell to develop in a uterus of the female non-human mammal; and d) identifying germline transmission in a genetically modified non-human mammal of a progeny of the pregnant female of step c).

In the present application, the animal may comprise a pig. The pig may be any species of pig inhibited in the art, including but not limited to those as defined above. For example, the pig may be selected from a Bama pig, a Wuzhishan pig and/or a Landrace pig.

In another aspect, the present application provides an animal model prepared according to said method for constructing an animal model in the present application, wherein said animal does not express said β4GalNT2 gene and/or β-1,4-N-acetylgalactosaminyl transferase 2. In some cases, the animal may not express the GGTA1 gene and/or αGal. In some cases, the animal may not express the CMAH gene and/or Neu5Gc.

In the present application, the non-expression of the β4GalNT2 gene, GGTA1 and/or CMAH generally refers to the insertion, interruption or deletion of the nucleotide sequence of the gene, or involves the reduction or loss of the gene's function of transcription and mRNA translation to a precursor or mature protein, or the encoding or non-encoding of a polypeptide having fewer amino acid residues than the endogenous amino acid sequence. In the present application, the non-expression of β-1,4-N-acetylgalactosaminyl transferase 2, αGal and/or Neu5Gc involves the reduction or elimination of activity or level.

The expression level of a gene or protein can be detected by a variety of methods, including methods at an RNA level (including mRNA quantification by reverse transcriptase polymerase chain reaction (RT-PCR) or by Southern blotting, in situ hybridization) and methods at a protein level (including histochemistry, immunoblotting analysis, and in vitro binding studies). Furthermore, the expression level of a gene of interest may be quantified by the ELISA technique that is well known to those skilled in the art. Quantitative measurement may be done by using a number of standard assays. For example, a transcript level may be measured by using the RT-PCR and the hybridization methods including RNase protection, Southern blotting analysis, RNA dot analysis. Immunohistochemical staining and flow cytometry as well as Western blot analysis may also be used to assess the presence of the β4GalNT2 gene and/or β-1,4-N-acetylgalactosaminyl transferase 2, the GGTA1 gene and/or aGal, and/or, the CMAH gene and/or Neu5Gc.

In another aspect, the present application provides a method for preparing an animal model. The method may comprise: a) providing the animal model of the present application; and b) mating the animal model obtained in step a) with another animal, or performing in vitro fertilization on the animal model obtained in step a), or further performing gene editing on the animal model obtained based on step a), or transplanting a human tissue and cell into the animal model obtained in step a), and performing screening to obtain an animal model.

In another aspect, the present application provides an animal model prepared according to said method for preparing an animal model. For example, the animal may include a pig.

In another aspect, the present application provides a cell or cell line or primary cell culture, wherein the cell or cell line or primary cell culture may be derived from the animal model of the present application or a progeny thereof. A cell culture may be isolated from a non-human mammal, or prepared from cell cultures established using standard cell transfection techniques with the same constructs.

In another aspect, the present application provides a tissue or organ or a culture thereof, wherein the tissue or organ or the culture thereof may be derived from the animal model or a progeny thereof.

In another aspect, the present application provides a CRISPR/Cas9 system for specifically targeting and knocking out a β4GalNT2 gene. The CRISPR/Cas9 system may comprise the use of a DNA sequence containing the gRNA specifically targeting the β4GalNT2 gene in the present application.

In another aspect, the present application provides a nucleic acid molecule kit capable of specifically targeting a β4GalNT2 gene. The kit may comprise the gRNA specifically targeting the β4GalNT2 gene.

In another aspect, the present application provides a complete set of nucleic acid molecules capable of specifically targeting a β4GalNT2 gene, wherein the complete set of nucleic acid molecules may comprise the sgRNA specifically targeting the β4GalNT2 gene and an mRNA of Cas9 nuclease.

In another aspect, the present application provides an application of said cell or cell line or primary cell culture or said tissue or organ or the culture thereof in development of an organ and/or tissue transplantation product, or as a model system for pharmacological, immunological and medical researches, or in validation, evaluation or study of immune rejection.

In another aspect, the present application provides an application of said animal model in development of an organ and/or tissue transplantation product, or as a model system for pharmacological, immunological and medical researches.

The organ and/or tissue transplantation product comprises cells, tissues or organs that are derived from different species and to be transplanted, implanted or infused into a receptor subject. The transplantation with a human as a receptor is particularly contemplated. The transplantation product may be isolated from a transgenic animal with reduced expression of αGal, β-1,4N-acetylgalactosaminyl transferase 2 and Neu5Gc.

The organ and/or tissue transplantation product may be isolated from a prenatal, neonatal, immature or fully mature transgenic animal. A transplantation material may be used as a temporary or permanent organ substitute for a human subject in need of organ transplantation.

In another aspect, the present application provides an application of said animal model in the aspect of validating, evaluating or studying immune rejection. An immune rejection occurs when a transplanted tissue, organ, cell or material is not accepted by the body of the receptor. In the immune rejection, the immune system of the receptor attacks the transplanted material. There are various types of immune rejections that may occur individually or together. Immune rejection includes, but is not limited to, a hyperacute rejection (HAR), an acute humoral xenograft rejection (AHXR), thrombocytopenia, an acute humoral rejection, a hyperacute vascular rejection, an antibody-mediated rejection, and a graft-versus-host disease.

Any method for evaluating, assessing, analyzing, measuring, quantifying or determining rejection-related symptoms known in the art can be used with the composition, kit and method of the present application. A method for analyzing symptoms associated with the immune rejection may include, but are not limited to, a laboratory assessment including CBC with platelet count, a coagulation study, a liver function test, flow cytometry, immunohistochemistry, standard diagnostic criteria, an immunological method, western blotting, immunoblotting, microscopy, confocal microscopy, transmission electron microscopy, an IgG binding assay, an IgM binding assay, an expression assay, a creatinine assay and endosome isolation.

Not wishing to be bound by any particular theory, the following examples are merely to illustrate the fusion protein, preparation methods and uses and the like according to the present application, and are not intended to limit the scope of the present invention.

### EXAMPLES

### Example 1 Construction of CRISPR/Cas9 Vector

First, according to the DNA sequence of a GGTAUCMAH/β4GalNT2 gene, sgRNAs targeting the GGTA1, CMAH and β4GalNT2 genes were synthesized, and a GGTA1-CRISPR/Cas9 vector, a CMAH-CRISPR/Cas9 vector and a β4GalNT2-CRISPR/Cas9 vector were respectively constructed by using pX330 (Add gene plasmid 423230) as a backbone plasmid.

First, according to the sequences of pig GGTA1, CMAH and β4GalNT2 genes published in Genbank, an exon 3 of the GGTA1 gene, an exon 6 of the CMAH gene and an exon 8 of β4GalNT2 gene were selected as CRISPR/Cas9 targets; according to the cas9 target design principle with G as a 5′-terminal and a PAM sequence (NGG) as a 3′-terminal, a sgRNA sequence targeting GGTA1 was designed as GAAAATAATGAATGTCAA (SEQ ID NO: 9), a sgRNA sequence targeting CMAH is designed as GAGTAAGGTACGTGATCTGT (SEQ ID NO: 10), a sgRNA1 sequence targeting β4GalNT2D is designed as GGTAGTACTCACGAACACTC (SEQ ID NO: 6), and a sgRNA2 sequence targeting β4GalNT2 is designed as CTACCCTTTCTTGCCCAGAG (SEQ ID NO: 7). A sgRNA sequence with a 5′-terminal phosphorylated oligonucleotide chain was synthesized.

The sgRNA sequence was cloned into the pX330 backbone vector (for targeting of the β4GalNT2 gene, a cloned fragment was U6 promoter-β4GalNT2/sgRNA1-gRNA backbone-U6 promoter-β4GalNT2/sgRNA2-gRNA backbone when the two sgRNAs were in the same vector), with the specific steps as follows.
1. 1 μg of pX330 plasmid was digested with a restriction enzyme BbsI;
2. the digested pX330 plasmid was isolated by being processed in an agarose gel (with a concentration of 1%, i.e., 1 g of agarose gel added to 100 mL of electrophoresis buffer), and the digested product was purified and recovered with a gel recovery kit (QIAGEN);
3. a synthesized sgRNA with a 5′-terminal phosphorylated oligonucleotide chain was annealed according to the following procedures:
   37° C. 30 min
   95° C. for 5 min and then cooling to 25° C. at a rate of 5° C./min;
4. a ligation reaction was initiated according to the following system: reacting 10 min at room temperature

| pX330 digested with BbsI in step 2 | 50 | ng |
|---|---|---|
| 5′-terminal phosphorylated oligonucleotide (l:250 v/v, | 1 | μL |
| diluted using sterile water) annealed in step 3 | | |
| 2x quick ligation buffer (NEB) | 5 | μL |
| ddH₂O | making up the | |
| | system to 10 μL | |
| subtotal | 10 | μL |
| quick ligation buffer (NEB) | 1 | μL |
| Total | 11 | μL |

5. the ligation system was treated with a plasmid-safe exonuclease to remove mis-ligated plasmids:

| | | |
|---|---|---|
| ligation reaction system obtained in step 4 | 11 | μL |
| 10x plasmid-safe buffer (NEB) | 1.5 | μL |
| 10 mM ATP | 1.5 | μL |
| plasmid-safe exonuclease (NEB) | 1 | μL |
| Total | 15 | μL |

reacting at 37° C. for 30 min

6. Transformation
   (1) 50 μL of competent cells (TIANGEN) was placed in an ice bath;
   (2) 15 μL of the solution with the mis-ligated plasmids removed from step 5 was added to a centrifuge tube containing the competent cells, mixed and let stand in the ice bath for 30 min;
   (3) the competent cells undergoing the ice bath for 30 min was placed into a water bath at 42° C., held for 60-90 s, and then quickly transferred into the ice bath for cooling for 2-3 min;
   (4) 900 μL of sterile LB culture medium (without antibiotics) was added to the centrifuge tube, mixed, and placed on a 37° C. shaker for shaking culture at 150 rpm for 45 min; and
   (5) the centrifuge tube was placed into a centrifuge at 12,000 rpm for 5 min, then 900 μL of supernatant was discarded, competent cell pellets was resuspended in the remaining 100 μL of supernatant, and then the resuspended competent cells were added to an LB solid agar culture medium containing corresponding antibiotics, and evenly spread by using a sterile spreader; and the LB solid agar culture medium with the spread competent cells was placed upside down in the 37° C. incubator for culturing for 12-16 hours; and
7. the plasmids were extracted by miniprep and sequenced. It was identified that the targeting plasmids were constructed successfully.

The CRISPR/Cas9 targeting vectors were obtained, and respectively named GGTA1-CRISPR/Cas9 (FIG. 1, with a full nucleotide sequence as set forth in SEQ ID NO: 4), CMAH-CRISPR/Cas9 (FIG. 2, with a full nucleotide sequence as set forth in SEQ ID NO: 5) and β4GalNT2-CRISPR/Cas9 (FIG. 3, with a full nucleotide sequence as set forth in SEQ ID NO: 3), with their nucleotide sequences as shown.

### Example 2 Construction of GGTA1/CMAH/β4GalNT2 Triple Knockout Clone

The GGTA1-CRISPR/Cas9 targeting vector, the CMAH-CRISPR/Cas9 targeting vector and the β4GalNT2-CRISPR/Cas9 targeting vector were simultaneously transfected into embryonic fibroblasts of male Landrace pigs, and single-cell clones with triple gene knockout were obtained by G418 screening. The specific steps were as follows.

2.1 Recovery of Primary Pig Fibroblasts
1. The cryopreserved primary pig fibroblasts was taken from liquid nitrogen and thawed in a 37° C. water bath.
2. The thawed cells were transferred into a 15 mL sterile centrifuge tube, then 3 mL of cell culture medium was added, and centrifuge was carried out at 1500 rpm for 5 min. The formula of cell complete culture medium included: 16% fetal bovine serum (Gibco)+84% DMEM culture medium (Gibco), with 16% and 84% representing volume percents.
3. The supernatant was discarded; 2 mL of the complete culture medium was added to resuspend cell pellets; then the resuspended cells were spread into a 6 cm cell culture dish; 2 mL of the complete culture medium was added; and the dish was placed in a 37° C. constant-temperature incubator containing 5% CO₂ (volume percent) for culturing.
4. When the cells grew to cover about 90% of the bottom of the dish, 0.05% (5 g/100 mL) trypsin was used to digest the cells; then, the complete culture medium was added to terminate the digestion; the cell suspension was transferred into a 15 mL centrifuge tube, and centrifuged at 1500 rpm for 5 min; the supernatant was discarded; the cells were resuspended in 2 mL of the complete culture medium and counted; and the total count of cells was adjusted to 1.5×10⁶ for the next nucleofection experiment.

2.2 The Primary Pig Fibroblasts were Co-Transfected with the Constructed GGTA1-CRISPR/Cas9 Targeting Vector, the CMAH-CRISPR/Cas9 Targeting Vector and the β4GalNT2-CRISPR/Cas9 Targeting Vector as Well as tdTomato Plasmids (Clontech, PT4069-5).

The nucleofection experiment were carried out using a mammalian fibroblast nucleofection kit (Lonza) and a Lonza Nucleofactor™ 2b.
1. A nucleofection reaction solution was prepared, with a system as follows:

| | |
|---|---|
| basic nucleofection solution | 82 μL |
| supplements | 8 μL |

2. The three constructed plasmids and the Tdtomato plasmids were added, respectively at a mass ratio of 5:1, to 100 μL of nucleofection reaction solution obtained in step 1, and mixed well, taking care to prevent air bubbles during the process.
3. The cell suspension prepared in 2.1 was washed twice with the Dulbecco's phosphate-buffered saline (DPBS, Gibco), and digested at 37° C. for 2 min. The digestion was terminated with the DMEM complete culture medium containing 10% fetal bovine serum by volume. The resultant was centrifuged at 1500 rpm for 5 min, and the supernatant was discarded. The cells were resuspended in the nucleofection reaction solution containing the plasmids from step 2. Air bubbles should be avoided during the resuspension.
4. The nucleofection system was carefully added into an electroporation cuvette supplied with the kit, taking care to prevent air bubbles. First, the electroporation cuvette containing 100 μL of PBS was placed in a cuvette holder of the Lonza nucleofector. A U023 nucleofection program was selected and set up. Then, the electroporation cuvette containing cells was electroporated for transfection, after which the liquid in the electroporation cuvette was immediately and gently aspirated in a super clean bench, transferred to 1 mL of DMEM complete culture medium containing 16% fetal bovine serum by volume, and mixed gently.
5. Several culture dishes (10 cm) containing 8 mL of complete culture medium were prepared. The cell suspension after nucleofection was pipetted into the culture dish containing the complete culture medium, and mixed well. The number of cells was observed under a microscope and counted, such that about 50-60 cells in the culture dish were present in the next field of view of the microscope. For the remaining dishes, the cell suspension was added thereto according to this final amount. The cells were mixed well and then placed in a 37° C. constant-temperature incubator containing 5% CO₂ for cultivation.

2.3 Screening of Triple-Gene Knockout Cell Lines
1. The cells obtained in 2.2 were cultured for 24 hours, and then the cell culture medium was replaced with a complete culture medium containing 1 mg/mL G418. The cells were placed in the 37° C. constant-temperature incubator containing 5% CO₂ for culture. The cell culture medium was changed every 2 to 3 days, during which the concentration of G418 was gradually reduced according to a cell growth condition to a final concentration of 0.3 mg/mL. After about 10 to 14 days of culture, G418-resistant monoclonal cell lines gradually grew in the culture dish.
2. The cell lines were picked using a clone ring. The picked monoclonal cell lines were seeded into a 24-well plate plated with 0.3 mg/mL G418 complete culture medium. The plate was placed in the 37° C. constant-temperature incubator containing 5% CO₂ for culture. The cell culture medium was changed every 2-3 days.
3. When the cells in the wells of the 24-well plate grew to cover the bottoms of the wells, the cells were digested with trypsin and collected. ⅘ of the cells were seeded into a 12-well plate or 6-well plate (depending on the amount of cells) containing 0.3 mg/mL G418 complete culture medium; and the remaining ⅕ of the cells remained in the 24-well plate for continued culture.
4. After the cells in the 12-well plate or 6-well plate grew to cover the bottoms of the wells, the cells were digested with 0.05% (5 g/100 mL) trypsin and collected, and then cryopreserved using a cryopreservation solution (90% fetal bovine serum+10% DMSO, by volume).

2.4 Gene Identification of Triple-Gene Knockout Cell Lines
1. After the cells in the 24-well plate grew to cover the bottoms of the wells, the cells were digested with 0.05% (5 g/100 mL) trypsin and collected. Then, 25 ml of NP-40 lysis buffer was added to the cells to lyse the cells for extracting the genomic DNA of the cells, with a lysis procedure including: 55° C. for 60 min—95° C. for 5 min—4° C. After the reaction ended, the genomic DNA was stored at −20° C.;
2. The corresponding PCR primers were designed according to the target information of the GGTAUCMAH/β4GalNT2 gene. The PCR primer sequences were as follows:

GGTA1
a forward primer was:
(SEQ ID NO: 11)
5′-CCTTAGTATCCTTCCCAACCCAGAC-3′,
a reverse primer was:
(SEQ ID NO: 12)
5′-GCTTTCTTTACGGTGTCAGTGAATCC-3′,
and
the length of a PCR product of interest was 428 bp;
CMAH
a forward primer was:
(SEQ ID NO: 13)
5′-CTTGGAGGTGATTTGAGTTGGG-3′,
a reverse primer was:
(SEQ ID NO: 14)
5′-GATTTTCTTCGGAGTTGAGGGC-3′,
and
the length of a PCR product of interest was 485 bp;
p4GalNT2
a forward primer was:
(SEQ ID NO: 15)
5′-CCCAAGGATCCTGCTGCC-3′,
a reverse primer was:
(SEQ ID NO: 8)
5′-CGCCGTGTAAAGAAACCTCC-3′,
and
the length of a PCR product of interest was 406 bp.

3. The GGTA1/CMAH/β4GalNT2 target gene was amplified by PCR, with a PCR reaction system including:

| | | |
|---|---|---|
| Cell genome DNA | 2 | μL |
| GGTA1 forward primer (10 pM) | 1 | μL |
| GGTA1 reverse primer (10 pM) | 1 | μL |
| 2X Taq enzyme premix | 25 | μL |
| ddH₂O | 21 | μL |
| Total | 50 | μL |

reaction conditions:

| | | | | | |
|---|---|---|---|---|---|
| Step 1 | 95° C. | 5 min | | | |
| Step 2 | 95° C. | | 30 s | | |
| | 64° C. | | 30 s | {close oversize brace} | 35 cycles |
| | 72° C. | | 45 s - | | |
| Step 3 | 72° C. | 7 min | | | |
| Step 4 | 4° C. | ∞ | | | |

The amplification of the CMAH target gene was the same as the above steps; the amplification of the β4GalNT2 target gene was the same as the above steps.
4. The PCR reaction product was subjected to agarose gel electrophoresis (1%, i.e., 1 g of agarose gel added to 100 mL of electrophoresis buffer). After the electrophoresis, the band of interest was cut under ultraviolet light, and then recovered using a gel recovery kit (QIAGEN). The concentration of the recovered PCR product was measured using NanoDrop 200.
5. The recovered PCR product was linked to a T vector using the TAKARA pMD™ 18-T Vector Cloning Kit. The reaction system of the T vector included:

| | | |
|---|---|---|
| pMD 18-T vector | 1 | μL |
| Gel recovered PCR | 81.7 | ng* |

| | |
|---|---|
| product | |
| ddH₂O | making up the system to 10 μL |

*Note:
Based on the dosage of 0.1-0.3 pM required for Insert DNA (the gel recovered PCR product in this case) in the instruction manual of the TAKARA pMD ™18-T Vector Cloning Kit, 0.2 pM was selected in this study. The dosage was calculated as follows: the amount of Insert DNA used (ng) = the value of nmol × 660 × the bp number of Insert DNA.

The reaction condition for T vector linking was: 16° C. for 30 min.
6. The T vector linked product obtained in step 5 was transformed using the competent cells (TIANGEN), and after the transformation, the competent cells were spread on Amp-resistant LB agar solid culture medium, and cultured in the 37° C. constant-temperature incubator overnight;
   Monoclonal colonies were picked from the medium cultured overnight and sent to a sequencing company for sequencing. Then, the sequencing results were aligned with the target information of GGTA1/CMAH/β4GalNT2 to determine whether the cell line is the GGTA1/CMAH/β4GalNT2 gene knockout cell line.

### Example 3 Knockout of 134GalNT2 Using Single sgRNA

According to the method of Example 2, a β4GalNT2 CRISPR/Cas9 targeting vector was constructed using the sgRNA (SEQ ID NO: 6) for β4GalNT2, and a CCTA1 CRISPR/Cas9 targeting vector was constructed using the sgRNA (SEQ ID NO: 9) for GGTA1; Bama pig and Wuzhishan pig cell clones with the double knockout of β4GalNT2 and GGTA1 were constructed, and sequenced to obtain the knockout efficiency and the genotypes of double-knockout cell clones.

The results are shown in Table 1 to Table 4. Among the 35 cryopreserved clones of Bama pigs, β4GalNT2 was successfully knocked out in 7 clones, and β4GalNT2 and GGTA1 were successfully and simultaneously knocked out in 7 clones. The genotypes of 7 clones with double-gene knockout were shown in Table 2. Among the 68 cryopreserved clones of Wuzhishan pigs, β4GalNT2 was successfully knocked out in 12 clones, and β4GalNT2 and GGTA1 were successfully and simultaneously knocked out in 9 clones. The genotypes of 9 clones with double-gene knockout were shown in Table 4. The efficiency of knocking out the β4GalNT2 using the single sgRNA for β4GalNT2 was 21.88% (Bama pigs) and 17.65% (Wuzhishan pigs). The double-knockout efficiency for the β4GalNT2 and GGTA1 was 21.88% (Bama pigs) and 13.24% (Wuzhishan pigs).

TABLE 1
Double knockout efficiency of Bama pigs

| | Number of | | Number of | |
|---|---|---|---|---|
| | β4GalNT2 | | double-gene | |
| Number of | knockout | β4GalNT2 | knockout | Double-gene |
| frozen clones | clones | knockout | clones | knockout |
| (pcs) | (pcs) | efficiency | (pcs) | efficiency |
| 32 | 7 | 21.88% | 7 | 21.88% |

TABLE 2
Genotypes of double-knockout cell clones of Bama pigs

| | | |
|---|---|---|
| | GGTA1 | β4GalNT2 |
| No. | knockout | knockout |

| | | | | |
|---|---|---|---|---|
| 3 | −2 | bp | −10 | bp |
| 18 | +l | bp (T) | +l-17 | bp |
| 24 | +l | bp (C) | −1 | bp (A) |
| 27 | +l-4 | bp | +1 | bp (A) |
| 41 | +l | bp (T) | −10 | bp |
| 42 | +1 | bp (T) | −10 | bp |
| 68 | +1 | bp (T) | +1 | bp (A) |

TABLE 3
Double knockout efficiency of Wuzhishan pigs

| | Number of | | Number of | |
|---|---|---|---|---|
| | p4GalNT2 | | double-gene | |
| Number of | knockout | p4GalNT2 | knockout | Double-gene |
| frozen clones | clones | knockout | clones | knockout |
| (pcs) | (pcs) | efficiency | (pcs) | efficiency |
| 68 | 12 | 17.65% | 9 | 13.24% |

TABLE 4
Genotypes of double-knockout cell clones of Wuzhishan pigs

| | | |
|---|---|---|
| No. | GGTA1 knockout | β4GalNT2 knockout |

| 4 | +1 bp (C), | −14 bp | +1 bp (A), | −2 bp |
|---|---|---|---|---|
| 13 | +1 | bp (C) | −1 bp (A), | −4 bp |
| 18 | −3 | bp (ATG) | +1 | bp (A) |
| 20 | −17 | bp | +1 bp, −1 bp (A), −5 bp | |
| 37 | −33 bp, −26 bp, +1 bp (C) | | +1 bp (C), | −10 bp |
| 40 | +1 | bp (C) | +1 bp (C), | −1 bp (A) |
| 43 | +1 | bp (C) | −10 bp, −4 bp, −3 bp | |
| 45 | +1 | bp (C) | +6-17 | bp |
| 56 | +1 | bp (C) | −2 | bp (AC) |

### Example 4 Knockout of 134GalNT2 Using Two sgRNAs

According to the method of Example 2, a β4GalNT2 CRISPR/Cas9 targeting vector was constructed using two sgRNA (SEQ ID NOs: 6 and 7) for β4GalNT2, a CCTA1 CRISPR/Cas9 targeting vector was constructed using the sgRNA (SEQ ID NO: 9) for GGTA1, and a CMAH CRISPR/Cas9 targeting vector was constructed using the sgRNA (SEQ ID NO: 10) for CMAH; and Landrace pig cell clones with the triple knockout of β4GalNT2, GGTA1 and CMAH were constructed, and sequenced to obtain the knockout efficiency and the genotypes of triple-knockout cell clones.

The results are shown in Table 5 to Table 7. Among the 59 cryopreserved clones of male Landrace pigs, β4GalNT2 was successfully knocked out in 18 clones, and β4GalNT2, GGTA1 and CMAH were successfully and simultaneously knocked out in 15 clones. The genotypes of 15 clones with triple-gene knockout were shown in Table 6. Among the 51 cryopreserved clones of male Landrace pigs, β4GalNT2 was successfully knocked out in 20 clones. The efficiency of knocking out the β4GalNT2 using two sgRNAs for β4GalNT2 was 30.51% (male Landrace pigs) and 39.21% (female landrace pigs). Compared with Example 3 in which one sgRNA was used, the knockout efficiency was increased by using two sgRNAs to knock out the β4GalNT2. The triple knockout efficiency for β4GalNT2, GGTA1 and CMAH was 25.42% (male landrace pigs), which was higher than the double-gene knockout efficiency (21.88%) in Example 2, indicating that the sgRNA of the present application not only achieved triple gene knockout, but also exhibited the knockout efficiency higher than that of the double-gene knockout.

TABLE 5
Efficiency of male knockout in Landrace pigs

| | Number of | | Number of | |
|---|---|---|---|---|
| | β4GalNT2 | | triple-gene | |
| Number of | knockout | β4GalNT2 | knockout | Triple-gene |
| frozen clones | clones | knockout | clones | knockout |
| (pcs) | (pcs) | efficiency | (pcs) | efficiency |
| 59 | 18 | 30.51% | 15 | 25.42% |

TABLE 6
Genotypes of triple-knockout cell clones of Landrace pigs

| | GGTA1 | | β4GalNT2 | | CMAH | |
|---|---|---|---|---|---|---|
| No. | knockout | | knockout | | knockout | |
| 2 | −1 | bp(T) | −81 | bp | −1 bp(G), −5 | |
| | | | | | bp(GATCA) | |
| 5 | −1 bp(T), +1 | | −80 bp, | −86 bp | −2 | bp(CA) |
| | bp(T), −37 bp | | | | | |
| 7 | +1 | bp(C) | −80 bp, | −81 bp | −10 | bp |
| 17 | +1 bp (C), | +1 bp(T) | −86 bp, −80 | | +2(GT) −5 | |
| | | | bp, −140 bp | | bp, −18 bp | |
| 18 | −6 bp, | +1 bp(T) | −80 | bp | −2 bp(GA), −12 | |
| | | | | | bp, −8 bp | |
| 22 | −4 bp(ATGT), | | −80 bp, | −81 bp | −1 | bp(G) |
| | C A | | | | | |
| 25 | −14 bp, | +1 bp(T) | −80 bp, | −15 bp | −5 | bp(GATCA) |

| 26 | +1 bp(G), +1 | | −80 bp, G→A | | −18 bp, −5 |
|---|---|---|---|---|---|
| | bp(T), −1 bp(G) | | 3(ACA) +l(T) bp | | bp(GATCA), −2 |
| | | | | | bp(GA), −9 bp |
| 28 | +1 bp (C), +1 | | −80 bp, | G→A | −9 bp, −1 bp |
| | bp(T), −14 bp | | | | (G), −1 bp(A) |
| 30 | −9 | bp | −80 bp, | +1 bp(T) | −1 bp(G), −l |
| | | | | | bp(A) |
| 35 | −5 bp(GAATG), +1 | | −80 bp, −l | | −1 bp(G), −5 |
| | bp(C) | | (A) +l(T) bp | | bp(GATCA) |
| 37 | −1 | bp(G) | −80 | bp | −1 bp(A), −4 |
| | | | | | bp(GATC) |

| | | | | |
|---|---|---|---|---|
| 39 | −1 | bp(G) | −80 bp, −2 | −2 bp(CA) , −4 |
| | | | (AC) −2(CT) bp | bp(GATC), −21 |
| | | | | bp +l bp (C) |

| | | | | | |
|---|---|---|---|---|---|
| 43 | +1 bp(T), +1 | −85 bp, | −83 bp | −2 | bp(GA) |
| | (T) −3(CAA) bp | | | | |

| | | | | | |
|---|---|---|---|---|---|
| 56 | −1 bp(T), | −2 bp(GT) | −3 | bp(TCC) | −1 bp(G), −2 |
| | | | | | bp(CA) |

TABLE 7
Knockout efficiency of female Landrace pigs

| | Number of | |
|---|---|---|
| Number of | β4GalNT2 | β4GalNT2 |
| cryopreserved | knockout | knockout |
| clones | clones (pcs) | efficiency |
| 51 | 20 | 39.21% |

What is claimed is:

1. A gRNA specifically targeting a β4GalNT2 gene, wherein said gRNA specifically binds to a nucleotide sequence as set forth in any one of SEQ ID NOs. 1-2.

2. The gRNA according to claim 1, wherein said gRNA comprises a nucleotide sequence as set forth in SEQ ID NO. 16.

3. The gRNA according to claim 1, wherein said gRNA comprises a nucleotide sequence as set forth in any one of SEQ ID NOs. 6-7.

4. The gRNA according to claim 1, wherein said sgRNA comprises 5′-(X)n-said nucleotide sequence as set forth in SEQ ID NO.16-a backbone sequence-3′, with X being a base selected from any one of A, U, C, and G, and n being any integer from 0 to 15.

5. The gRNA according to claim 1, wherein said gRNA is a single-strand guide RNA sgRNA.

6. A gRNA combination, comprising the gRNA specifically targeting the β4GalNT2 gene of claim 1, a gRNA specifically targeting a GGTA1 gene, and a gRNA specifically targeting a CMAH gene.

7. The gRNA combination according to claim 6, wherein said gRNA specifically targeting the GGTA1 gene comprises a nucleotide sequence as set forth in SEQ ID NO. 9, and/or said gRNA specifically targeting the CMAH gene comprises a nucleotide sequence as set forth in SEQ ID NO. 10.

8. (canceled)

9. (canceled)

10. (canceled)

11. (canceled)

12. (canceled)

13. (canceled)

14. (canceled)

15. A method for constructing an animal model, comprising administering at least two gRNAs specifically targeting a β4GalNT2 gene to an animal cell to knock out all or a portion of said β4GalNT2 gene, wherein said gRNA specifically binds to a nucleotide sequence as set forth in any one of SEQ ID NOs: 1-2.

16. The method according to claim 15, wherein said gRNA comprises a nucleotide sequence as set forth in SEQ ID NO. 16.

17. The method according to claim 15, wherein said gRNA specifically targeting the β4GalNT2 gene comprises a nucleotide sequence as set forth in any one of SEQ ID NOs. 6-7.

18. (canceled)

19. The method according to claim 15, wherein said gRNA is a single-strand guide RNA sgRNA.

20. (canceled)

21. The method according to claim 15, wherein said method further comprises administering a gRNA specifically targeting a GGTA1 gene to an animal cell to knock out all or a portion of said GGTA1 gene, and/or administering a gRNA specifically targeting a CMAH gene to an animal cell to knock out all or a portion of said CMAH gene.

22. The method according to claim 21, wherein said gRNA specifically targeting the GGTA1 gene comprises a nucleotide sequence as set forth in SEQ ID NO. and/or said gRNA specifically targeting the CMAH gene comprises a nucleotide sequence as set forth in SEQ ID NO. 10.

23. (canceled)

24. (canceled)

25. The method according to claim 15, wherein said method comprises the step of producing one or more single-strand breaks SSBs or double-strand breaks DSBs within or near said β4GalNT2 gene, said GGTA1 gene and/or said CMAH gene by using one or more deoxyribonucleic acid DNA endonucleases, such that all or a portion of one or more exons of said GGTA1 gene and/or said CMAH gene is/are deleted.

26. The method according to claim 25, wherein said DNA endonuclease comprises a Cas nuclease.

27. The method according to claim 26, wherein said Cas nuclease comprises a Cas9 nuclease, a homologue thereof, a recombinant of a naturally occurring molecule thereof, a codon-optimized version thereof, and/or a modified version thereof.

28. The method according to claim 15, comprising:
a) providing a cell comprising one or more in vitro transcription products comprising a vector of said gRNA;
b) culturing said cell in a culture solution;
c) transplanting said cultured cell into a fallopian tube of a receptor female non-human mammal, to allow said cell to develop in a uterus of said female non-human mammal; and
d) identifying germline transmission in a genetically modified non-human mammal of a progeny of said pregnant female of step c).

29. The method according to claim 15, wherein said animal comprises a pig.

30. (canceled)

31. (canceled)

32. (canceled)

33. (canceled)

34. (canceled)

35. (canceled)

36. A tissue, organ, a culture thereof, a cell or cell line or primary cell culture, wherein said tissue, organ, culture thereof, cell or cell line or primary cell culture is derived from said animal model prepared according to the method of claim 15.

37. (canceled)

38. A CRISPR/Cas9 system for specifically targeting and knocking out a β4GalNT2 gene, wherein a DNA sequence containing said gRNA specifically targeting the β4GalNT2 gene of claim 1.

39. (canceled)

40. (canceled)

41. (canceled)

42. (canceled)

43. (canceled)
